(19) United States
(12) Patent Application Publication
(10) Pub. No.: US 2010/0106198 A1
(43) Pub. Date: Apr. 29, 2010

(54) NERVE STIMULATING BONE SCREW

(75) Inventors: William Keith Adcox, Memphis, TN (US); Seth L. Neubardt, White Plaines, NY (US)

Correspondence Address: MEDTRONIC;Attn: Noreen Johnson - IP Legal Department, 2600 Sofamor Danek Drive, MEMPHIS, TN, 38132, US

(73) Assignee: Warsaw Orthopedic, Inc, Warsaw, IN (US)

(21) Appl. No.: 12/257,144

(22) Filed: Oct. 23, 2008

(51) Int. Cl. A61B 17/86; A61B 17/84; A61N 1/00; B25C 3/00

(52) U.S. Cl. 606/301; 606/331; 607/2; 81/44

(57) ABSTRACT

A nerve stimulating bone screw is disclosed and can include a shaft that can have a continuous thread formed thereon and a distal end. A head can be attached to the shaft. Also, the nerve stimulating bone screw can include a first conductor that can extend through the shaft. The first conductor can be insulated and can provide a signal at the distal end of the shaft. The signal can be configured to provide nerve stimulation at the distal end of the shaft of the nerve stimulating bone screw.

### FIELD OF THE DISCLOSURE

The present disclosure relates generally to bone screws. More specifically, the present disclosure relates to devices for stimulating nerves while installing bone screws.

### BACKGROUND

Certain spinal deformities, e.g., scoliosis, can be treated using a spinal fixation system. The spinal fixation system is a brace that can be installed along a spinal column in order to prevent further bending of the spine and to coax the spine into a relatively straighter position. The spinal fixation system can include a plurality of anchorage components. Further, one or more longitudinal elements can be installed along the anchorage components. Each anchorage component can be affixed to a corresponding vertebra using one or more bone screws, e.g., pedicle screws. The pedicle screws can be installed in a pedicle wall of a vertebra. Further, the pedicle screws are relatively strong and can provide stability for each anchorage component and the spinal fixation system.

During the installation of a spinal fixation system, great care should be used to avoid nerve impingement when installing the bone screws. Otherwise, a patient may be injured during the installation and suffer a loss of mobility of another part of the body due to the nerve impingement.

### BRIEF DESCRIPTION OF THE DRAWINGS

FIG. 1 is a plan view of a first embodiment of a nerve stimulating bone screw;

FIG. 2 is a cross-section view of the first embodiment of the nerve stimulating bone screw taken along line 2-2 in FIG. 1;

FIG. 3 is a plan view of a second embodiment of a nerve stimulating bone screw;

FIG. 4 is a cross-section view of the second embodiment of a nerve stimulating bone screw taken along line 4-4 in FIG. 3;

FIG. 5 is a plan view of a third embodiment of a nerve stimulating bone screw;

FIG. 6 is a cross-section view of the third embodiment of a nerve stimulating bone screw taken along line 6-6 in FIG. 5;

FIG. 7 is a plan view of a first embodiment of a nerve stimulating screwdriver;

FIG. 8 is a cross-section view of the first embodiment of the nerve stimulating screwdriver taken along line 8-8 in FIG. 7;

FIG. 9 is a plan view of a fourth embodiment of a nerve stimulating bone screw;

FIG. 10 is a cross-section view of the fourth embodiment of a nerve stimulating bone screw taken along line 10-10 in FIG. 9;

FIG. 11 is a plan view of a fifth embodiment of a nerve stimulating bone screw;

FIG. 12 is a cross-section view of the fifth embodiment of the nerve stimulating bone screw taken along line 12-12 in FIG. 11;

FIG. 13 is a plan view of a second embodiment of a nerve stimulating screwdriver;

FIG. 14 is a cross-section view of the second embodiment of the nerve stimulating screwdriver taken along line 14-14 in FIG. 13; and

FIG. 15 is a flow chart illustrating a method of treating a patient using a nerve stimulating bone screw.

### DETAILED DESCRIPTION OF THE DRAWINGS

A nerve stimulating bone screw is disclosed and can include a shaft that can have a continuous thread formed thereon and a distal end. A head can be attached to the shaft. Also, the nerve stimulating bone screw can include a first conductor that can extend through the shaft. The first conductor can be insulated and can provide a signal at the distal end of the shaft. The signal can be configured to provide nerve stimulation at the distal end of the shaft of the nerve stimulating bone screw.

In another embodiment, a method of treating a patient is disclosed and can include energizing a nerve stimulating bone screw. The nerve stimulating bone screw can provide a nerve stimulation signal at a distal end of the nerve stimulating bone screw. Further, the method can include advancing the nerve stimulating bone screw into tissue.

In yet another embodiment, a nerve stimulating bone screw is disclosed and can include a shaft that can have a continuous thread formed thereon and a distal end. A head can be coupled to the shaft. Also, a first conductor and a second conductor can extend along the shaft. The nerve stimulating bone screw can be configured to transmit a signal via the first conductor and to receive at least a portion of the signal via the second conductor.

In still another embodiment, a nerve stimulating screwdriver is disclosed and can include a shaft that can have a proximal end and a distal end. A handle can be coupled to the proximal end of the shaft. A screw engagement head can coupled to the distal end of the shaft. A first conductor can be incorporated into the screw engagement head. The nerve stimulating screwdriver can also include a power source and the power source can be configured to provide an electrical signal to the first conductor.

In another embodiment, a kit is disclosed and can include a nerve stimulating bone screw and a nerve stimulating screwdriver. The nerve stimulating screwdriver can be configured to deliver an electrical signal to the nerve stimulating bone screw.

### Description of a First Embodiment of a Nerve Stimulating Bone Screw

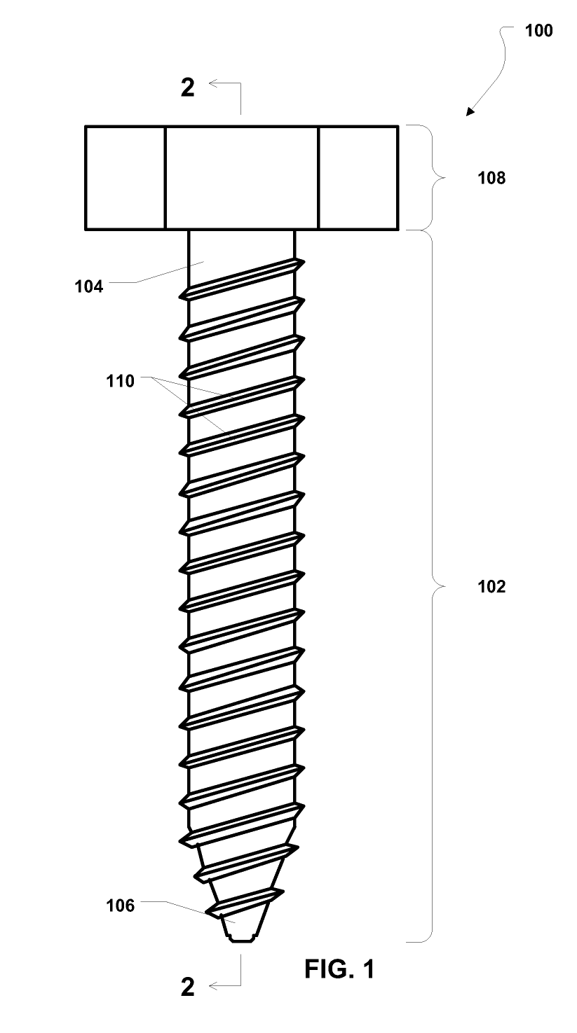

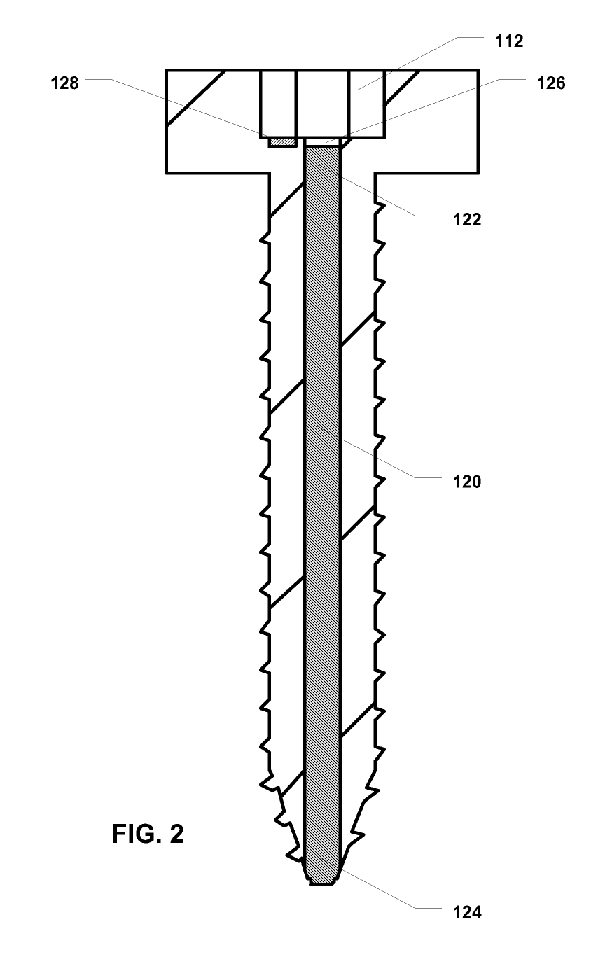

Referring to FIG. 1 and FIG. 2, a first embodiment of a nerve stimulating bone screw is shown and is generally designated 100. As shown in FIG. 1, the nerve stimulating bone screw 100 can include a shaft 102 having a proximal end 104 and a distal end 106. A head 108 can be attached to the proximal end 104 of the shaft 102. As shown in FIG. 1, the shaft 102 can include a continuous thread 110 formed along the length of the shaft 102 from the proximal end 104 to the distal end 102 of the shaft 102.

In a particular embodiment, the shaft 102 and the head 108 of the nerve stimulating bone screw 100 can be made from a dielectric material, i.e., a material that cannot conduct electricity. For example, the dielectric material can be a polymer material, a ceramic material, or a combination thereof. The polymer materials can include polyaramide materials, polyimid materials, polyamide materials, polyaryletherketone (PAEK) materials, or a combination thereof. The PAEK materials can include polyetherketone (PEK), polyetheretherketone (PEEK), polyetherketoneketone (PEKK), polyetherketoneetherketoneketone (PEKEKK), or a combination thereof. The ceramic materials can include alumina oxide, silica oxide, zirconium oxide, aluminum oxide, or a combination thereof.

Referring to FIG. 2, a cross-section of the nerve stimulating bone screw 100 is shown. As illustrated in FIG. 2, the head 108 of the nerve stimulating bone screw 100 can be formed with a tool engagement indentation 112. In a particular embodiment, the tool engagement indentation 112 can be generally hexagonal. Alternatively, the tool engagement indentation 112 can be a slot, a cross, a star shape, or some other shape that corresponds to an end of a tool such as a screwdriver.

As shown in FIG. 2, the nerve stimulating bone screw 100 can include a generally cylindrical conductor 120 therein. In a particular embodiment, the conductor 120 can include a proximal end 122 and a distal end 124. Further, the conductor 120 can extend from the distal end 106 of the shaft 102 through the proximal end 104 of the shaft 102 into the head 108 proximal to the tool engagement indentation 112. In a particular embodiment, the conductor 120 can be slightly distanced from the bottom of the tool engagement indentation 112 and can form a conductor engagement indentation 126 just below the tool engagement indentation 112.

Accordingly, an end of a tool can be inserted in the tool engagement indentation 112 and a conductor from the tool can extend into the conductor engagement indentation 126 and engage the conductor 120 in the nerve stimulating bone screw.

In a particular embodiment, the conductor 120 can be made from a conductive material, e.g., a metal material. The metal material can be a pure metal, a metal alloy, or a combination thereof. The pure metal can include aluminum, copper, gold, titanium, or a combination thereof. The metal alloy can include stainless steel, tungsten carbide, a cobalt-chrome-molybdenum alloy, a titanium alloy, or a combination thereof.

When the nerve stimulating bone screw 100 is engaged with a tool, e.g., a screwdriver, the proximal end 122 of the conductor 120 can engage a conductor within the tool and a power source within the tool can be used to provide an electric signal to the conductor 120 within the nerve stimulating bone screw 100. The electric signal can have a constant current that is less than or equal to fifty milliamps (50 ma). Since the shaft 102 and the head 108 of the nerve stimulating bone screw 100 are made from a dielectric material, only the conductor 120 can conduct electricity through the nerve stimulating bone screw 100. As such, the nerve stimulating bone screw 120 is configured so the distal end 124 of the conductor 120 can stimulate tissue proximate to the distal end 124 of the nerve stimulating bone screw 100. The shaft 102 of the nerve stimulating bone screw 100 can substantially insulate tissue surrounding the nerve stimulating bone screw 100 from the signal provided to the conductor 120.

FIG. 2 also illustrates that the nerve stimulating bone screw 100 can include a radio frequency identification (RFID) device 128 incorporated therein. For example, the RFID device 128 can be incorporated into the head 108 of the nerve stimulating bone screw 100. Further, the RFID device 128 can be passive and can be powered by an RFID reader placed near the nerve stimulating bone screw 100. Alternatively, the RFID device 128 can be active and the nerve stimulating bone screw 100 can include a power source, such as a battery (not shown). In a particular embodiment, the RFID device 128 can identify a size of the nerve stimulating bone screw 100; a date of manufacture of the nerve stimulating bone screw 100; a lot code associated with the production of the nerve stimulating bone screw 100; a type of material, or materials, comprising the nerve stimulating bone screw 100; or a combination thereof.

### Description of a Second Embodiment of a Nerve Stimulating Bone Screw

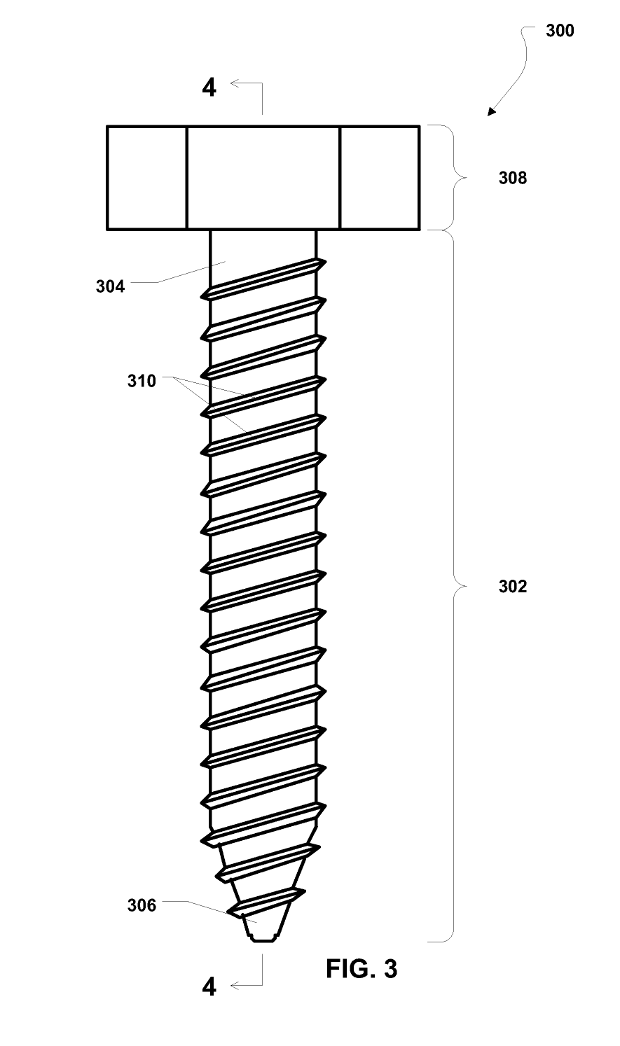

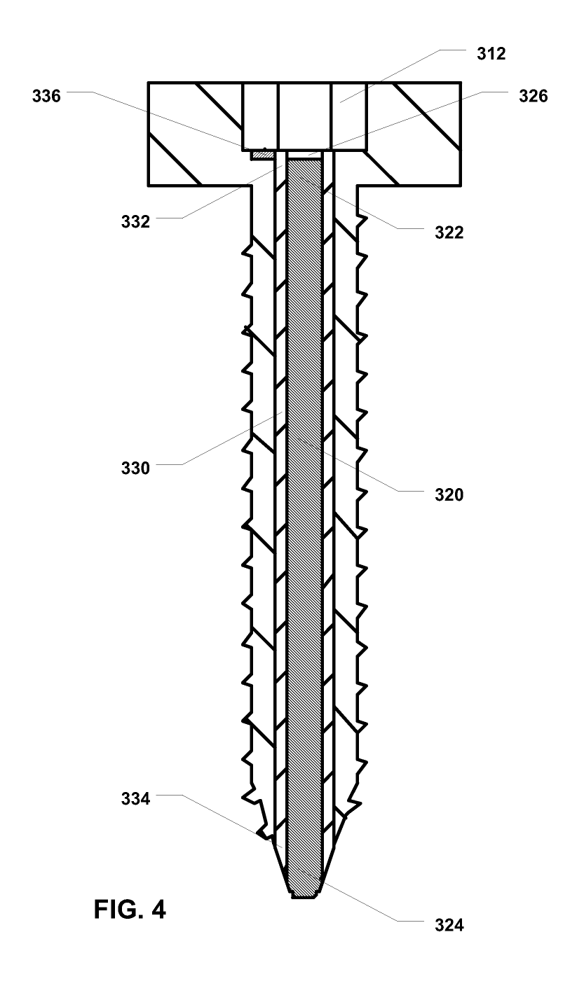

Referring to FIG. 3 and FIG. 4, a second embodiment of a nerve stimulating bone screw is shown and is generally designated 300. As shown in FIG. 3, the nerve stimulating bone screw 300 can include a shaft 302 having a proximal end 304 and a distal end 306. A head 308 can be attached to the proximal end 304 of the shaft 302. As shown in FIG. 3, the shaft 302 can include a continuous thread 310 formed along the length of the shaft 302 from the proximal end 304 to the distal end 302 of the shaft 302.

In a particular embodiment, the shaft 302 and the head 308 of the nerve stimulating bone screw 300 can be made from a metal material. The metal material can be a pure metal, a metal alloy, or a combination thereof. The pure metal can include titanium. The metal alloy can include stainless steel, tungsten carbide, a cobalt-chrome-molybdenum alloy, a titanium alloy, or a combination thereof.

Referring to FIG. 4, a cross-section of the nerve stimulating bone screw 300 is shown. As illustrated in FIG. 4, the head 308 of the nerve stimulating bone screw 300 can be formed with a tool engagement indentation 312. In a particular embodiment, the tool engagement indentation 312 can be generally hexagonal. Alternatively, the tool engagement indentation 312 can be a slot, a cross, a star shape, or some other shape that corresponds to an end of a tool, such as a screwdriver.

As shown in FIG. 4, the nerve stimulating bone screw 300 can include a generally cylindrical conductor 320 therein. In a particular embodiment, the conductor 320 can include a proximal end 322 and a distal end 324. Further, the conductor 320 can extend from the distal end 306 of the shaft 302 through the proximal end 304 of the shaft 302 into the head 308 proximal to the tool engagement indentation 312. In a particular embodiment, the conductor 320 can be slightly distanced from the bottom of the tool engagement indentation 312 and can form a conductor engagement indentation 326 just below the tool engagement indentation 312.

Accordingly, an end of a tool can be inserted in the tool engagement indentation 312 and a conductor from the tool can extend into the conductor engagement indentation 326 and engage the conductor 320 in the nerve stimulating bone screw 300.

In a particular embodiment, the conductor 320 can be made from a conductive material, e.g., a metal material. The metal material can be a pure metal, a metal alloy, or a combination thereof. The pure metal can include aluminum, copper, gold, titanium, or a combination thereof. The metal alloy can include stainless steel, tungsten carbide, a cobalt-chrome-molybdenum alloy, a titanium alloy, or a combination thereof.

As indicated in FIG. 4, an insulator 330 can surround the conductor 320. The insulator 330 can be generally hollow and generally cylindrical. Further, the insulator 330 can have a proximal end 332 and a distal end 334. In a particular embodiment, the insulator 330 can be made from a dielectric material, i.e., a material that cannot conduct electricity. For example, the dielectric material can be a polymer material, a ceramic material, or a combination thereof. The polymer materials can include polyaramide materials, polyimid materials, polyamide materials, polyaryletherketone (PAEK) materials, or a combination thereof. The PAEK materials can include polyetherketone (PEK), polyetheretherketone (PEEK), polyetherketoneketone (PEKK), polyetherketoneetherketoneketone (PEKEKK), or a combination thereof. The ceramic materials can include alumina oxide, silica oxide, zirconium oxide, aluminum oxide, or a combination thereof. Accordingly, the insulator 330 can insulate the conductor from the shaft 302 and head 308 of the nerve stimulating bone screw 300.

When the nerve stimulating bone screw 300 is engaged with a tool, e.g., a screwdriver, the proximal end 322 of the conductor 320 can engage a conductor within the tool and a power source within the tool can be used to provide an electric signal to the conductor 320 within the nerve stimulating bone screw 300. The electric signal can have a constant current that is less than or equal to fifty milliamps (50 ma). Since the conductor 320 is surrounded by an insulator 330, only the conductor 320 can conduct electricity through the nerve stimulating bone screw 300. As such, the nerve stimulating bone screw 300 is configured so the distal end 326 of the conductor 320 can stimulate tissue proximate to the distal end 306 of the shaft 302 of the nerve stimulating bone screw 300. The insulator 330 within the nerve stimulating bone screw 300 can substantially insulate tissue surrounding the nerve stimulating bone screw 300 from the signal provided to the conductor 320.

FIG. 4 also illustrates that the nerve stimulating bone screw 300 can include a radio frequency identification (RFID) device 336 incorporated therein. For example, the RFID device 336 can be incorporated into the head 308 of the nerve stimulating bone screw 300. Further, the RFID device 336 can be passive and can be powered by an RFID reader placed near the nerve stimulating bone screw 300. Alternatively, the RFID device 336 can be active and the nerve stimulating bone screw 300 can include a power source, such as a battery (not shown). In a particular embodiment, the RFID device 336 can identify a size of the nerve stimulating bone screw 300; a date of manufacture of the nerve stimulating bone screw 300; a lot code associated with the production of the nerve stimulating bone screw 300; a type of material, or materials, comprising the nerve stimulating bone screw 300; or a combination thereof.

### Description of a Third Embodiment of a Nerve Stimulating Bone Screw

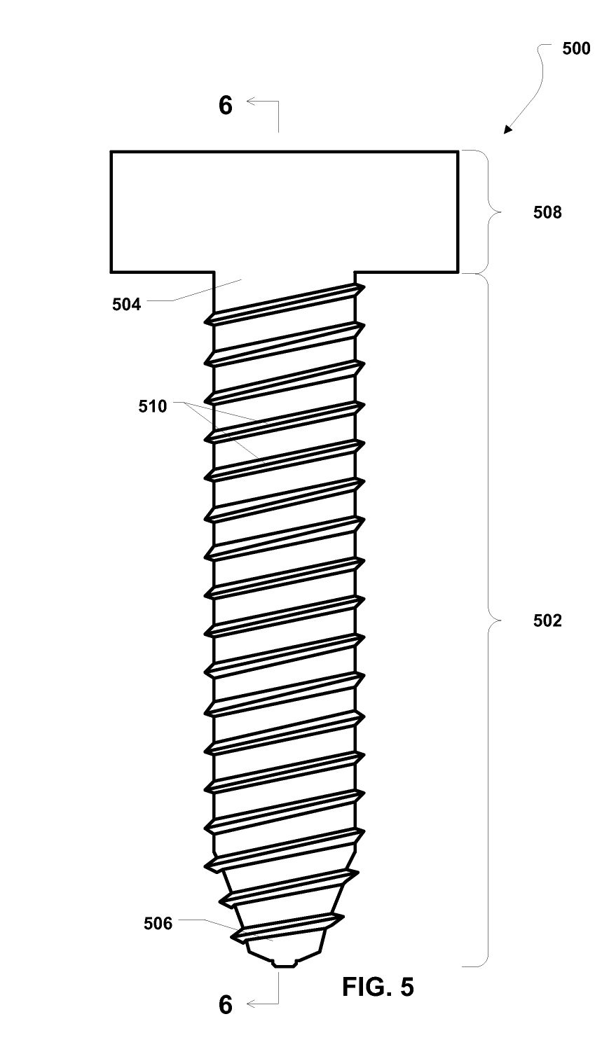

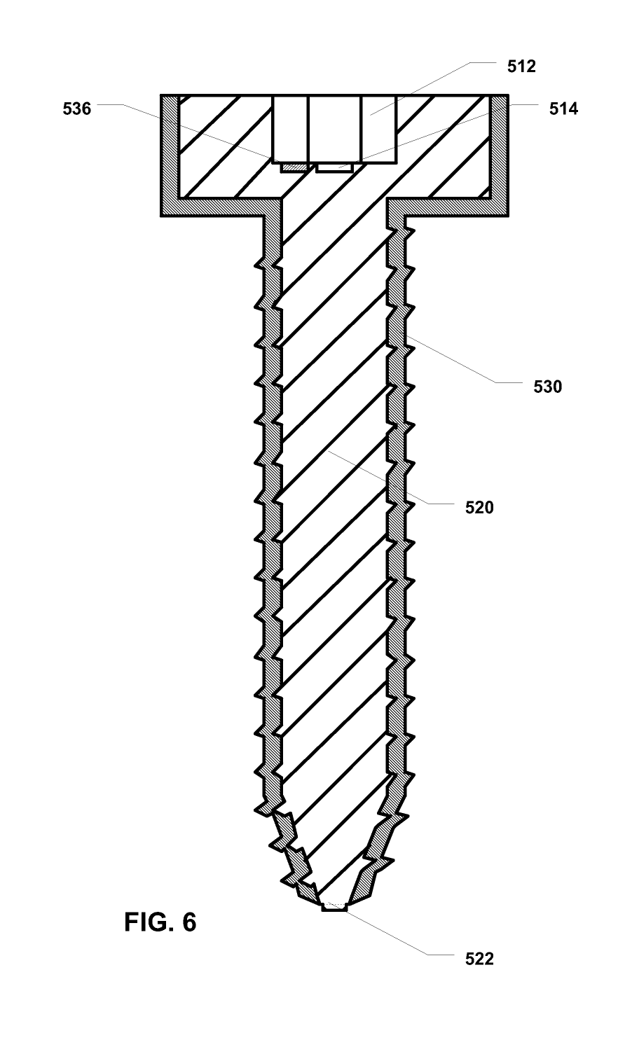

Referring to FIG. 5 and FIG. 6, a third embodiment of a nerve stimulating bone screw is shown and is generally designated 500. As shown in FIG. 5, the nerve stimulating bone screw 500 can include a shaft 502 having a proximal end 504 and a distal end 506. A head 508 can be attached to the proximal end 504 of the shaft 502. As shown in FIG. 5, the shaft 502 can include a continuous thread 510 formed along the length of the shaft 502 from the proximal end 504 to the distal end 502 of the shaft 502.

Referring to FIG. 6, a cross-section of the nerve stimulating bone screw 500 is shown. As illustrated in FIG. 6, the head 508 of the nerve stimulating bone screw 500 can be formed with a tool engagement indentation 512. In a particular embodiment, the tool engagement indentation 512 can be generally hexagonal. Alternatively, the tool engagement indentation 512 can be a slot, a cross, a star shape, or some other shape that corresponds to an end of a tool, such as a screwdriver. The head 508 of the nerve stimulating bone screw 500 can also be formed with a conductor engagement indentation 514 just below the tool engagement indentation 512. Accordingly, an end of a tool can be inserted in the tool engagement indentation 512 and a conductor from the tool can extend into the conductor engagement indentation 514

As further illustrated in FIG. 6, the nerve stimulating bone screw 500 can include a core 520 and an insulating layer 530 over the core 520. The core 520 can include a distal end 522 that extends through the insulating layer 530.

In a particular embodiment, the insulating layer 530 can be made from a dielectric material, i.e., a material that cannot conduct electricity. For example, the dielectric material can be a polymer material, a ceramic material, or a combination thereof. The polymer materials can include polyaramide materials, polyimid materials, polyamide materials, polyaryletherketone (PAEK) materials, or a combination thereof. The PAEK materials can include polyetherketone (PEK), polyetheretherketone (PEEK), polyetherketoneketone (PEKK), polyetherketoneetherketoneketone (PEKEKK), or a combination thereof. The ceramic materials can include alumina oxide, silica oxide, zirconium oxide, aluminum oxide, or a combination thereof.

In a particular embodiment, the core 520 can be made a conductive material, e.g., a metal material. The metal material can be a pure metal, a metal alloy, or a combination thereof. The pure metal can include aluminum, copper, gold, titanium, or a combination thereof. The metal alloy can include stainless steel, tungsten carbide, a cobalt-chrome-molybdenum alloy, a titanium alloy, or a combination thereof.

When the nerve stimulating bone screw 500 is engaged with a tool, e.g., a screwdriver, a conductor from the tool can extend into the conductor engagement indentation 514 and engage the core 520. A power source within the tool can be used to provide an electric signal to the core 520 of the nerve stimulating bone screw 500. The electric signal can have a constant current that is less than or equal to fifty milliamps (50 ma). Since the core 520 is surrounded by an insulator 530, only the core 520 can conduct electricity through the nerve stimulating bone screw 500. As such, the nerve stimulating bone screw 500 is configured so the distal end 522 of the core 520 can stimulate tissue proximate to the distal end 506 of the shaft 502 of the nerve stimulating bone screw 500. The insulating layer 530 around the core 520 of the nerve stimulating bone screw 500 can substantially insulate tissue surrounding the nerve stimulating bone screw 500 from the signal provided to the core 520.

FIG. 6 also illustrates that the nerve stimulating bone screw 500 can include a radio frequency identification (RFID) device 536 incorporated therein. For example, the RFID device 536 can be incorporated into the head 508 of the nerve stimulating bone screw 500. Further, the RFID device 536 can be passive and can be powered by an RFID reader placed near the nerve stimulating bone screw 500. Alternatively, the RFID device 536 can be active and the nerve stimulating bone screw 500 can include a power source, such as a battery (not shown). In a particular embodiment, the RFID device 536 can identify a size of the nerve stimulating bone screw 500; a date of manufacture of the nerve stimulating bone screw 500; a lot code associated with the production of the nerve stimulating bone screw 500; a type of material, or materials, comprising the nerve stimulating bone screw 500; or a combination thereof.

### Description of a First Embodiment of a Nerve Stimulating Screwdriver

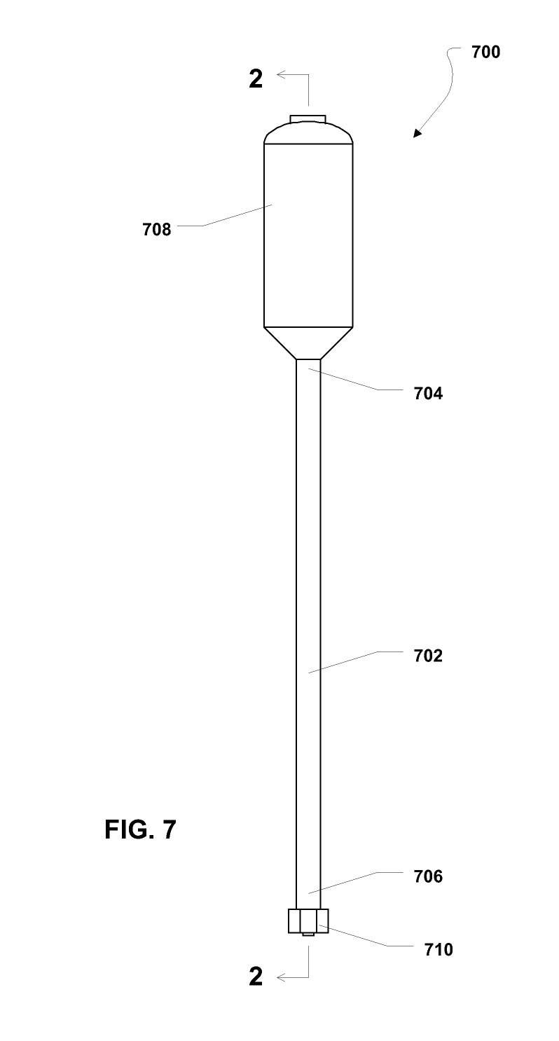

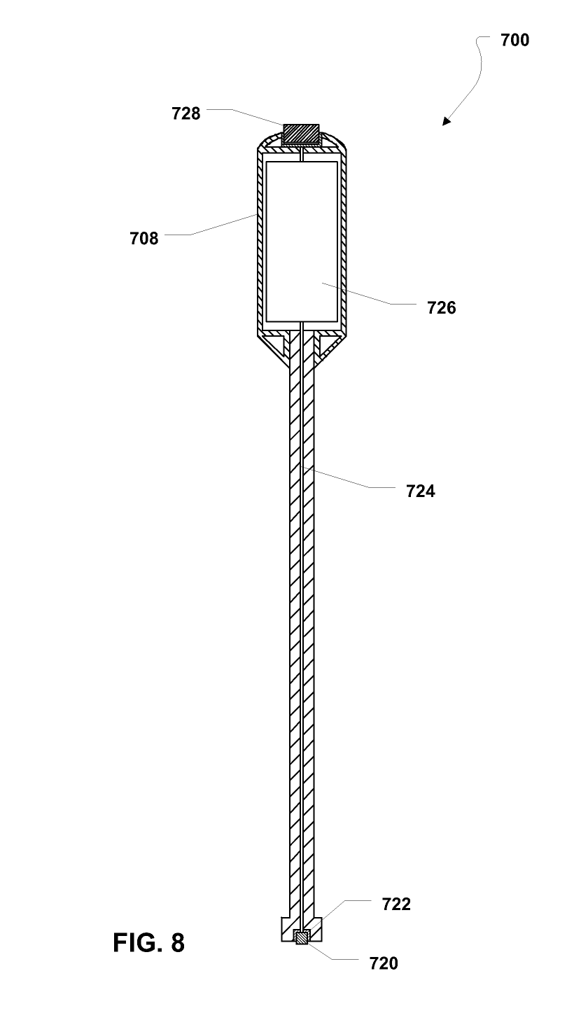

Referring to FIG. 7 and FIG. 8, a first embodiment of a nerve stimulating screwdriver is shown and is generally designated 700. As depicted, the nerve stimulating screwdriver 700 can include a shaft 702 having a proximal end 704 and a distal end 706. A handle 708 can be coupled to, or engaged with, the proximal end 704 of the shaft 702. Further, a screw engagement head 710 can be formed with, or coupled to, the distal end 706 of the shaft 702. In a particular embodiment, the screw engagement head 710 is configured to engage the head of a screw.

FIG. 8 indicates that a conductor 720 can be disposed within the screw engagement head 710. The conductor 720 can be surrounded by an insulator 722 and a wire 724 can extend from the conductor 720 to a power source 726 within the handle 708 of the nerve stimulating screwdriver 700. Also, a switch 728 can be incorporated into the handle 708 of the nerve stimulating screwdriver 700. In a particular embodiment, the switch 728 can be a push button, on/off switch and can be used to control the flow of power from the power source 726 to the wire 724 and in turn, the conductor 720.

When the nerve stimulating screwdriver 700 is engaged with a nerve stimulating bone screw, e.g., one of the nerve stimulating bone screws described herein, the conductor 720 within the screw engagement head 710 can engage a conductor within the nerve stimulating bone screw. Further, the nerve stimulating screwdriver 700 can be energized to deliver a signal to the nerve stimulating bone screw and the nerve stimulating bone screw can stimulate tissue as it is advanced into tissue by the nerve stimulating screwdriver 700.

### Description of a Fourth Embodiment of a Nerve Stimulating Bone Screw

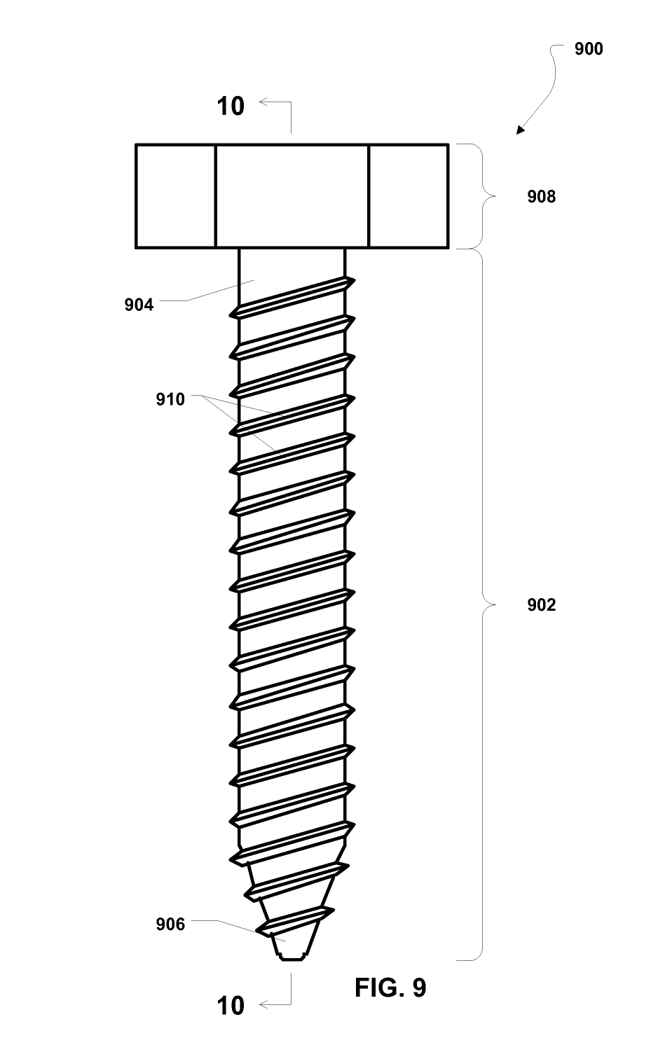

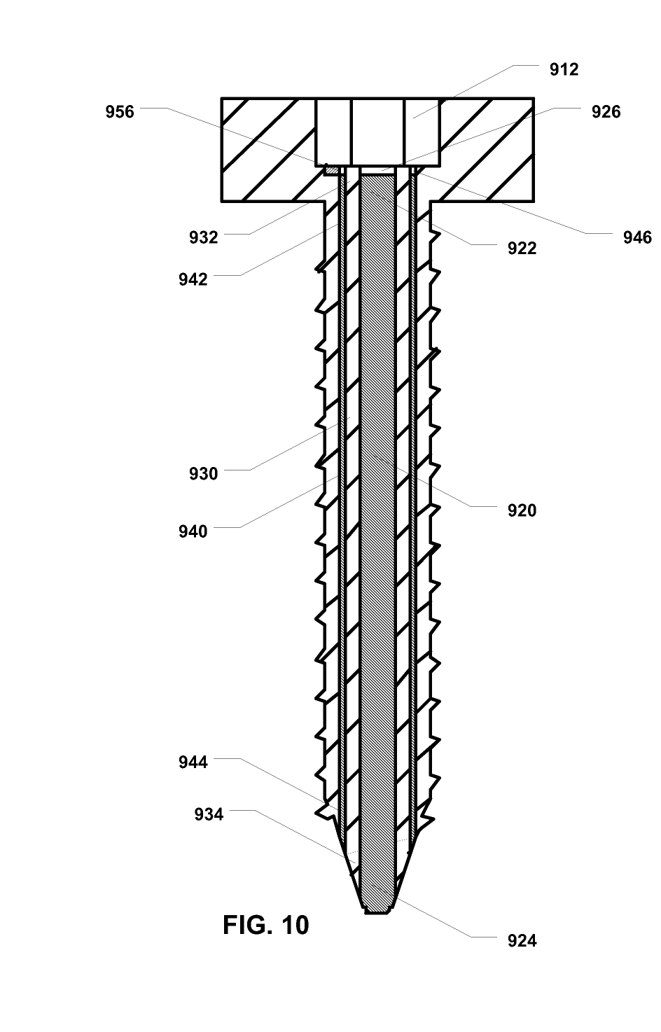

Referring to FIG. 9 and FIG. 10, a fourth embodiment of a nerve stimulating bone screw is shown and is generally designated 900. As shown in FIG. 9, the nerve stimulating bone screw 900 can include a shaft 902 having a proximal end 904 and a distal end 906. A head 908 can be attached to the proximal end 904 of the shaft 902. As shown in FIG. 9, the shaft 902 can include a continuous thread 910 formed along the length of the shaft 902 from the proximal end 904 to the distal end 902 of the shaft 902.

In a particular embodiment, the shaft 902 and the head 908 of the nerve stimulating bone screw 900 can be made from a dielectric material, i.e., a material that cannot conduct electricity. For example, the dielectric material can be a polymer material, a ceramic material, or a combination thereof. The polymer materials can include polyaramide materials, polyimid materials, polyamide materials, polyaryletherketone (PAEK) materials, or a combination thereof. The PAEK materials can include polyetherketone (PEK), polyetheretherketone (PEEK), polyetherketoneketone (PEKK), polyetherketoneetherketoneketone (PEKEKK), or a combination thereof. The ceramic materials can include alumina oxide, silica oxide, zirconium oxide, aluminum oxide, or a combination thereof.

Referring to FIG. 10, a cross-section of the nerve stimulating bone screw 900 is shown. As illustrated in FIG. 10, the head 908 of the nerve stimulating bone screw 900 can be formed with a tool engagement indentation 912. In a particular embodiment, the tool engagement indentation 912 can be generally hexagonal. Alternatively, the tool engagement indentation 912 can be a slot, a cross, a star shape, or some other shape that corresponds to an end of a tool such as a screwdriver.

As shown in FIG. 10, the nerve stimulating bone screw 900 can include a first conductor 920 therein. In a particular embodiment, the first conductor 920 can be generally cylindrical and can include a proximal end 922 and a distal end 924. Further, the first conductor 920 can extend from the distal end 906 of the shaft 902 through the proximal end 904 of the shaft 902 into the head 908 proximal to the tool engagement indentation 912. In a particular embodiment, the first conductor 920 can be slightly distanced from the bottom of the tool engagement indentation 912 and can form a first conductor engagement indentation 926 just below the tool engagement indentation 912.

FIG. 10 also indicates that an insulator 930 can surround the first conductor 920. The insulator 930 can be generally hollow and generally cylindrical. Further, the insulator 930 can include a proximal end 932 and a distal end 934. A second conductor 940 can surround the insulator 930. The second conductor 940 can also define a proximal end 942 and a distal end 944. In a particular embodiment, the second conductor 940 can be slightly distanced from the bottom of the tool engagement indentation 912 and can form a second conductor engagement indentation 946 just below the tool engagement indentation 912 and around the first conductor engagement indentation 926.

Accordingly, an end of a tool can be inserted in the tool engagement indentation 912 and a first conductor from the tool can extend into the conductor engagement indentation 926 and engage the first conductor 920 within the nerve stimulating bone screw 900. A second conductor from the tool can extend into the second conductor engagement indentation 946 and engage the second conductor 940 within the nerve stimulating bone screw 900.

In a particular embodiment, the conductors 920, 940 can be made from a conductive material, e.g., a metal material. The metal material can be a pure metal, a metal alloy, or a combination thereof. The pure metal can include aluminum, copper, gold, titanium, or a combination thereof. The metal alloy can include stainless steel, tungsten carbide, a cobalt-chrome-molybdenum alloy, a titanium alloy, or a combination thereof.

The insulator 930 can be made from a dielectric material, i.e., a material that cannot conduct electricity. For example, the dielectric material can be a polymer material, a ceramic material, or a combination thereof. The polymer materials can include polyaramide materials, polyimid materials, polyamide materials, polyaryletherketone (PAEK) materials, or a combination thereof. The PAEK materials can include polyetherketone (PEK), polyetheretherketone (PEEK), polyetherketoneketone (PEKK), polyetherketoneetherketoneketone (PEKEKK), or a combination thereof. The ceramic materials can include alumina oxide, silica oxide, zirconium oxide, aluminum oxide, or a combination thereof.

When the nerve stimulating bone screw 900 is engaged with a tool, e.g., a screwdriver, the proximal end 922 of the first conductor 920 can engage a first conductor within the tool. Further, the proximal end 942 of the second conductor 940 can engage a second conductor within the tool. A signal can be transmitted via the first conductor 920 within the nerve stimulating bone screw 900. Further, at least a portion of the signal can be received via the second conductor 940 within the nerve stimulating bone screw 900. The transmitted and received signal can be used to determine an impedance value and the impedance value can be used to determine the type of tissue in which the nerve stimulating bone screw 900 is advancing.

FIG. 10 also illustrates that the nerve stimulating bone screw 900 can include a radio frequency identification (RFID) device 956 incorporated therein. For example, the RFID device 956 can be incorporated into the head 908 of the nerve stimulating bone screw 900. Further, the RFID device 956 can be passive and can be powered by an RFID reader placed near the nerve stimulating bone screw 900. Alternatively, the RFID device 956 can be active and the nerve stimulating bone screw 900 can include a power source, such as a battery (not shown). In a particular embodiment, the RFID device 956 can identify a size of the nerve stimulating bone screw 900; a date of manufacture of the nerve stimulating bone screw 900; a lot code associated with the production of the nerve stimulating bone screw 900; a type of material, or materials, comprising the nerve stimulating bone screw 900; or a combination thereof.

### Description of a Fifth Embodiment of a Nerve Stimulating Bone Screw

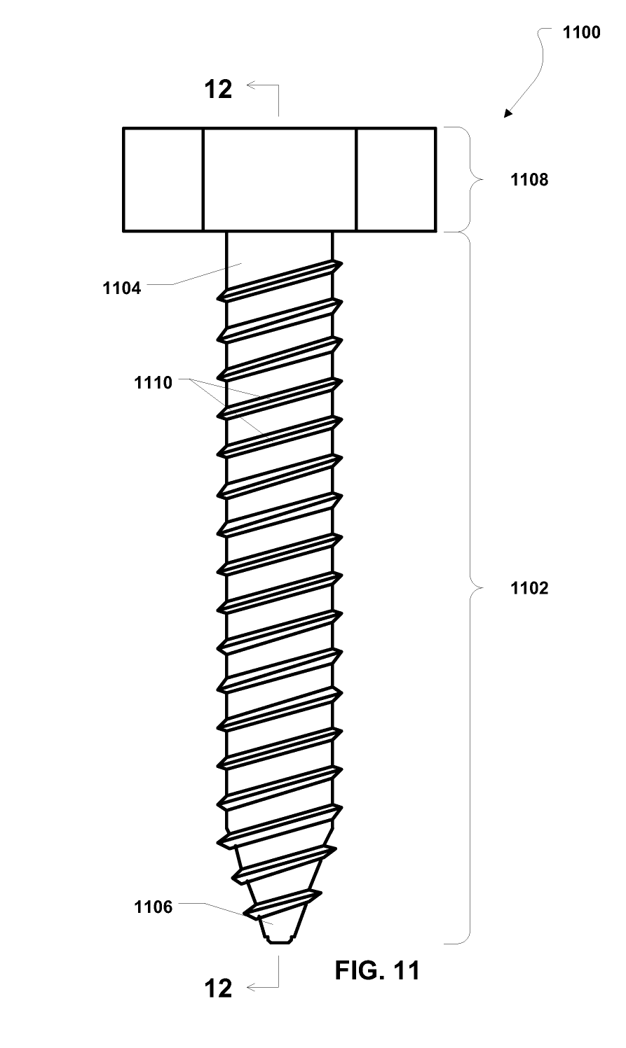

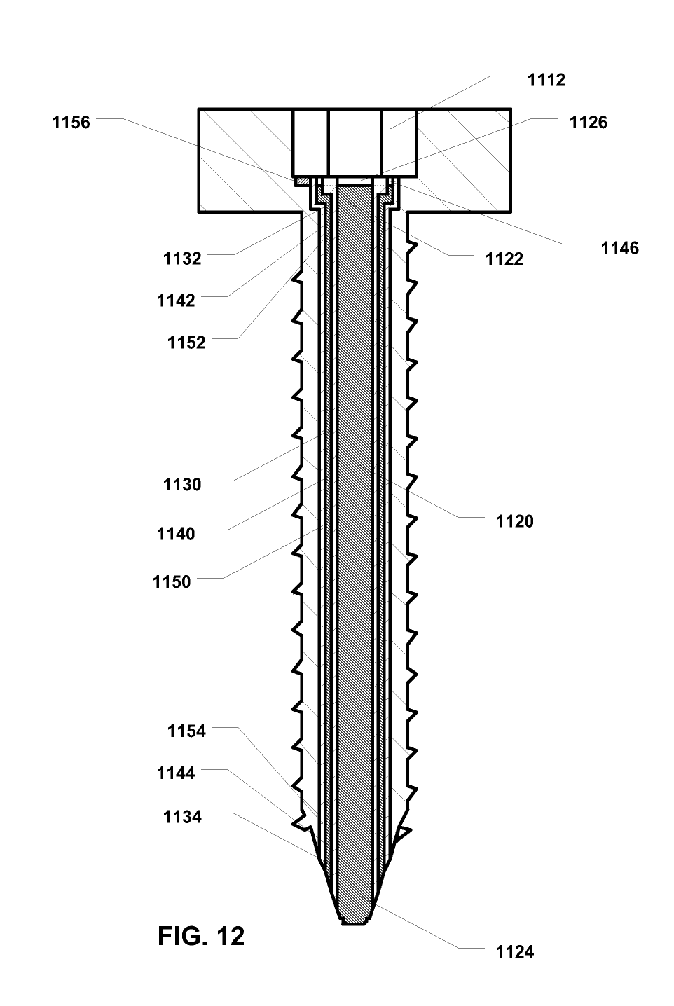

Referring to FIG. 11 and FIG. 12, a fifth embodiment of a nerve stimulating bone screw is shown and is generally designated 1100. As shown in FIG. 11, the nerve stimulating bone screw 1100 can include a shaft 1102 having a proximal end 1104 and a distal end 1106. A head 1108 can be attached to the proximal end 1104 of the shaft 1102. As shown in FIG. 11, the shaft 1102 can include a continuous thread 1110 formed along the length of the shaft 1102 from the proximal end 1104 to the distal end 1102 of the shaft 1102.

In a particular embodiment, the shaft 1102 and the head 1108 of the nerve stimulating bone screw 1100 can be made from a metal material. The metal material can be a pure metal, a metal alloy, or a combination thereof. The pure metal can include titanium. The metal alloy can include stainless steel, tungsten carbide, a cobalt-chrome-molybdenum alloy, a titanium alloy, or a combination thereof.

Referring to FIG. 12, a cross-section of the nerve stimulating bone screw 1100 is shown. As illustrated in FIG. 12, the head 1108 of the nerve stimulating bone screw 1100 can be formed with a tool engagement indentation 1112. In a particular embodiment, the tool engagement indentation 1112 can be generally hexagonal. Alternatively, the tool engagement indentation 1112 can be a slot, a cross, a star shape, or some other shape that corresponds to an end of a tool such as a screwdriver.

As shown in FIG. 12, the nerve stimulating bone screw 1100 can include a first conductor 1120 therein. In a particular embodiment, the first conductor 1120 can be generally cylindrical and can include a proximal end 1122 and a distal end 1124. Further, the first conductor 1120 can extend from the distal end 1106 of the shaft 1102 through the proximal end 1104 of the shaft 1102 into the head 1108 proximal to the tool engagement indentation 1112. In a particular embodiment, the first conductor 1120 can be slightly distanced from the bottom of the tool engagement indentation 1112 and can form a first conductor engagement indentation 1126 just below the tool engagement indentation 1112.

FIG. 12 also indicates that a first insulator 1130 can surround the first conductor 1120. The insulator 1130 can be generally hollow and generally cylindrical. Further, the insulator 1130 can include a proximal end 1132 and a distal end 1134. A second conductor 1140 can surround the insulator 1130. The second conductor 1140 can also define a proximal end 1142 and a distal end 1144. In a particular embodiment, the second conductor 1140 can be slightly distanced from the bottom of the tool engagement indentation 1112 and can form a second conductor engagement indentation 1146 just below the tool engagement indentation 1112 and around the first conductor engagement indentation 1126. A second insulator 1150 can surround the second conductor 1140. The second insulator 1150 can be generally hollow and generally cylindrical. Further, the second insulator 1150 can include a proximal end 1152 and a distal end 1154.

Accordingly, an end of a tool can be inserted in the tool engagement indentation 1112 and a first conductor from the tool can extend into the conductor engagement indentation 1126 and engage the first conductor 1120 within the nerve stimulating bone screw 1100. A second conductor from the tool can extend into the second conductor engagement indentation 1146 and engage the second conductor 1140 within the nerve stimulating bone screw 1100.

In a particular embodiment, the conductors 1120, 1140 can be made from a conductive material, e.g., a metal material. The metal material can be a pure metal, a metal alloy, or a combination thereof. The pure metal can include aluminum, copper, gold, titanium, or a combination thereof. The metal alloy can include stainless steel, tungsten carbide, a cobalt-chrome-molybdenum alloy, a titanium alloy, or a combination thereof.

The insulators 1130, 1150 can be made from a dielectric material, i.e., a material that cannot conduct electricity. For example, the dielectric material can be a polymer material, a ceramic material, or a combination thereof. The polymer materials can include polyaramide materials, polyimid materials, polyamide materials, polyaryletherketone (PAEK) materials, or a combination thereof. The PAEK materials can include polyetherketone (PEK), polyetheretherketone (PEEK), polyetherketoneketone (PEKK), polyetherketoneetherketoneketone (PEKEKK), or a combination thereof. The ceramic materials can include alumina oxide, silica oxide, zirconium oxide, aluminum oxide, or a combination thereof.

When the nerve stimulating bone screw 1100 is engaged with a tool, e.g., a screwdriver, the proximal end 1122 of the first conductor 1120 can engage a first conductor within the tool. Further, the proximal end 1142 of the second conductor 1140 can engage a second conductor within the tool. A signal can be transmitted via the first conductor 1120 within the nerve stimulating bone screw 1100. Further, at least a portion of the signal can be received via the second conductor 1140 within the nerve stimulating bone screw 1100. The transmitted and received signal can be used to determine an impedance value and the impedance value can be used to determine the type of tissue in which the nerve stimulating bone screw 1100 is advancing.

FIG. 12 also illustrates that the nerve stimulating bone screw 1100 can include a radio frequency identification (RFID) device 1156 incorporated therein. For example, the RFID device 1156 can be incorporated into the head 1108 of the nerve stimulating bone screw 1100. Further, the RFID device 1156 can be passive and can be powered by an RFID reader placed near the nerve stimulating bone screw 1100. Alternatively, the RFID device 1156 can be active and the nerve stimulating bone screw 1100 can include a power source, such as a battery (not shown). In a particular embodiment, the RFID device 1156 can identify a size of the nerve stimulating bone screw 1100; a date of manufacture of the nerve stimulating bone screw 1100; a lot code associated with the production of the nerve stimulating bone screw 1100; a type of material, or materials, comprising the nerve stimulating bone screw 1100; or a combination thereof.

### Description of a Second Embodiment of a Nerve Stimulating Screwdriver

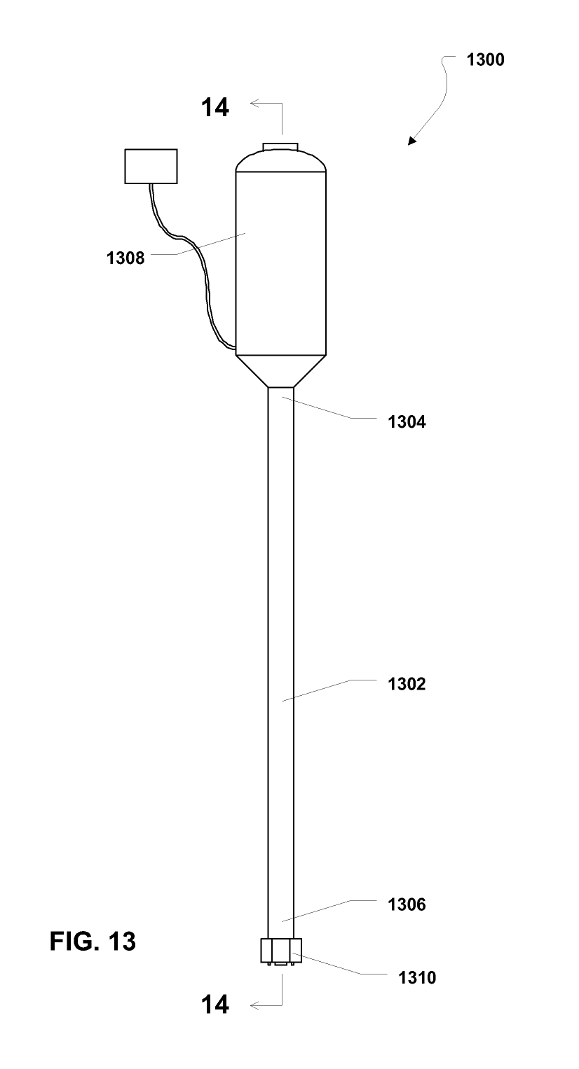

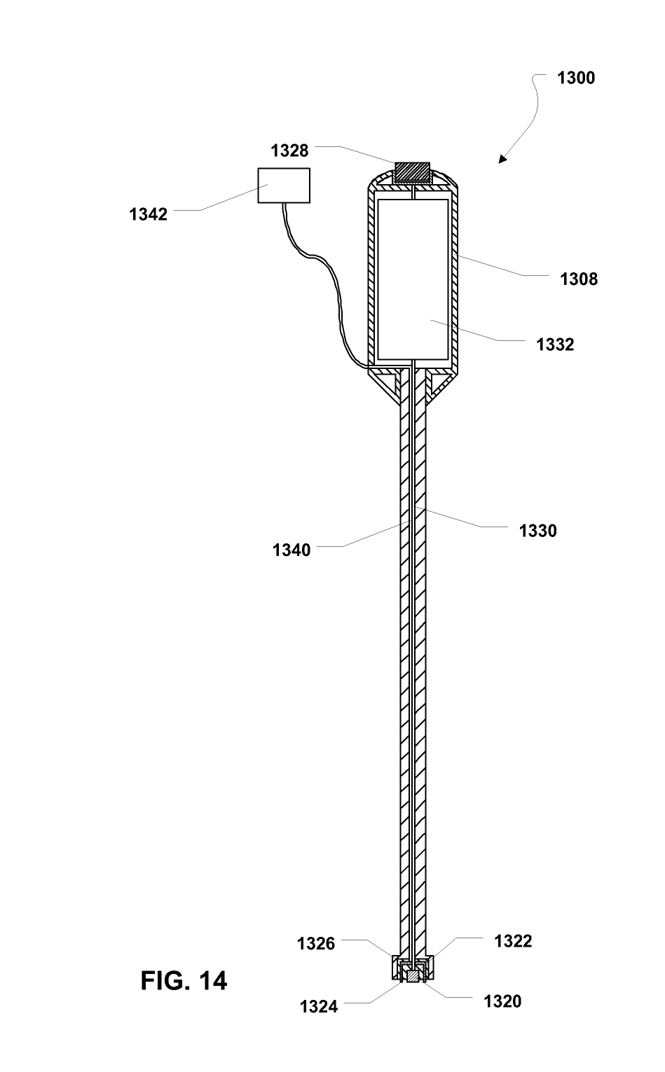

Referring to FIG. 13 and FIG. 14, a first embodiment of a nerve stimulating screwdriver is shown and is generally designated 1300. As depicted, the nerve stimulating screwdriver 1300 can include a shaft 1302 having a proximal end 1304 and a distal end 1306. A handle 1308 can be coupled to, or engaged with, the proximal end 1304 of the shaft 1302. Further, a screw engagement head 1310 can be formed with, or coupled to, the distal end 1306 of the shaft 1302. In a particular embodiment, the screw engagement head 1310 is configured to engage the head of a screw.

FIG. 14 indicates that a first conductor 1320 can be disposed within the screw engagement head 1310. The first conductor 1320 can be surrounded by a first insulator 1322. Additionally, a second conductor 1324 can surround the first insulator 1322 and a second insulator 1326 can surround the second conductor 1324. A first wire 1330 can extend from the first conductor 1320 to a power source 1332 within the handle 1308 of the nerve stimulating screwdriver 1300. Also, a switch 1334 can be incorporated into the handle 1308 of the nerve stimulating screwdriver 1300. In a particular embodiment, the switch 1334 can be a push button, on/off switch and can be used to control the flow of power from the power source 1332 to the first wire 1330 and in turn, the first conductor 1320.

A second wire 1340 can connect the second conductor 1324 to a processor 1342. A signal can be transmitted via the first wire 1330. At least a portion of the signal can be received via the second wire 1340. The processor 1342 can process the signal received via the second wire 1340 in order to determine an impedance value associated with the signal. In a particular embodiment, the processor 1342 can be external to the nerve stimulating screwdriver 1300. Alternatively, the processor 1342 can be within the handle 1308 of the nerve stimulating screwdriver 1300. Also, the processor 1342 can be connected to a display device, e.g., a liquid crystal display (LCD), a light emitting diode (LED), or other display device.

When the nerve stimulating screwdriver 1300 is engaged with a nerve stimulating bone screw, e.g., one of the nerve stimulating bone screws described herein, the first conductor 1320 within the screw engagement head 1310 can engage a first conductor within the nerve stimulating bone screw. The second conductor 1324 within the screw engagement head 1320 can engage a second conductor within the nerve stimulating bone screw.

### Description of a Method of Treating a Patient

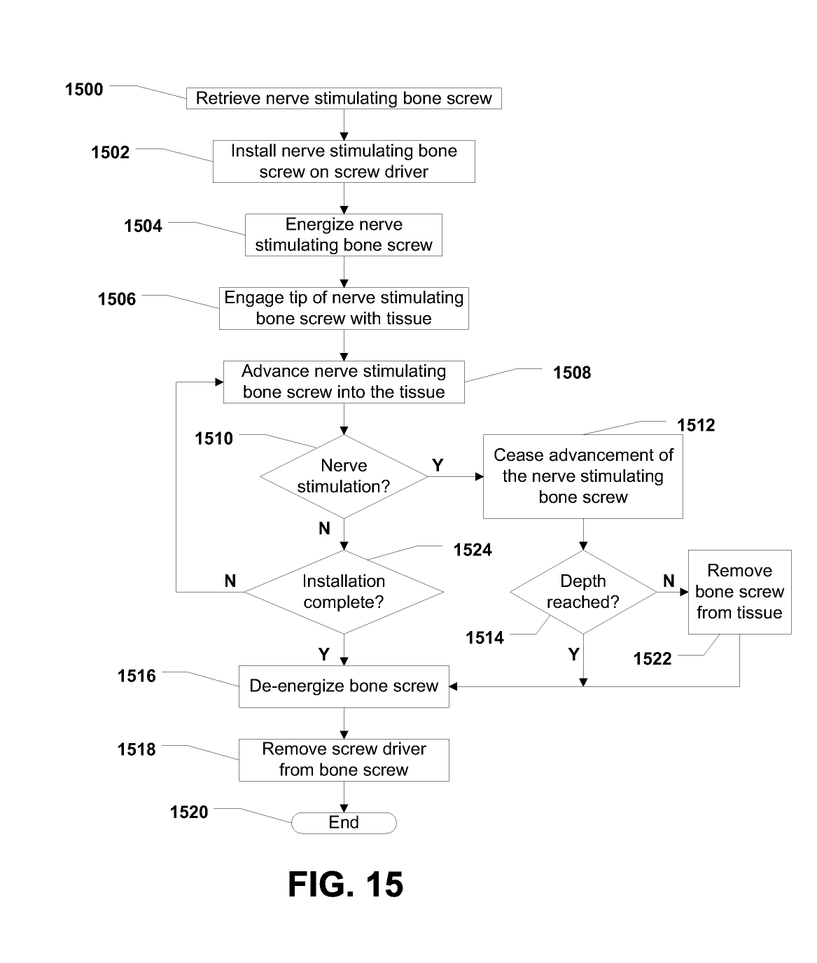

FIG. 15 is a flow chart illustrating a method of treating a patient using a nerve stimulating bone screw.

Moving to block 1504, the nerve stimulating bone screw can be energized. Further, at block 1506, the tip of the nerve stimulating bone screw can be engaged with tissue. At block 1508, the nerve stimulating drill can be advanced into the tissue. Proceeding to decision step 1510, it can be determined whether nerve stimulation has occurred. This determination can be made using electromyography. More specifically, this determination can be made by monitoring a location on the patient, e.g., a muscle, that corresponds to the nerve or nerves at the location being drill. When nerve stimulation occurs, the corresponding muscle or muscles contract in response to the electrical stimulation.

At decision step 1510, if nerve stimulation occurs, the method can move to block 1512, and the advancement of the nerve stimulating bone screw can be ceased. Thereafter, at decision step 1514, it can be determined whether an acceptable screw depth is reached. If so, the method can proceed to block 1516 and the nerve stimulating bone screw can be de-energized. At block 1518, the nerve stimulating screwdriver can be removed from the nerve stimulating bone screw can be removed from the surgical drill. The method can then end at state 1520.

Returning to decision step 1514, if an acceptable screw depth is not reached, the method can proceed to block 1522 and the nerve stimulating bone screw can be removed from the tissue. The nerve stimulating bone screw can be removed by reversing the direction of use of the nerve stimulating screwdriver. After the nerve stimulating bone screw is removed, the method can continue to block 1516 and continue as described herein.

Returning to decision step 1510, if nerve stimulation does not occur, the method can move to decision step 1524 and it can be determined whether installation of the nerve stimulating bone screw is complete, i.e., whether the nerve stimulating bone screw is installed to a desired depth is reached. If installation is not complete, the method can return to block 1508 and continue as described herein. On the other hand, if the installation of the nerve stimulating bone screw is complete, the method can continue to block 1516 and continue as described herein.

During use, the nerve stimulating bone screw may also be used to determine the type of tissue into which the nerve stimulating bone screw is advancing. For example, the nerve stimulating bone screw can transmit and receive a signal. The impedance of the signal can be measured and used to determine if the tip of the nerve stimulating bone screw is advancing into hard tissue, e.g., bone, soft tissue, e.g., flesh, or subcutaneous fluid.

### CONCLUSION

With the configuration of structure described above, the nerve stimulating bone screw provides a device that can be used for electromyography (EMG) and impedance measurements. For example, the nerve stimulating bone screw can provide a monopolar electric signal to tissue while the nerve stimulating bone screw is advancing into the tissue. When a nerve is stimulated, an EMG response can occur and advancement of the nerve stimulating bone screw can be altered to prevent damage to the nerve.

Additionally, the nerve stimulating bone screw can deliver a bipolar signal, i.e., the nerve stimulating bone screw can transmit a signal via a first conductor and receive at least a portion of the signal at a second conductor. A processor coupled to the nerve stimulating bone screw can measure the impedance of the signal and the impedance value can be used to determine if the nerve stimulating bone screw is advancing into hard tissue, soft tissue, or fluid.

The above-disclosed subject matter is to be considered illustrative, and not restrictive, and the appended claims are intended to cover all such modifications, enhancements, and other embodiments that fall within the true spirit and scope of the present invention. Thus, to the maximum extent allowed by law, the scope of the present invention is to be determined by the broadest permissible interpretation of the following claims and their equivalents, and shall not be restricted or limited by the foregoing detailed description.

What is claimed is:

1. A nerve stimulating bone screw, comprising:
a shaft having a continuous thread formed thereon and a distal end;
a head attached to the shaft; and
a first conductor extending through the shaft, wherein the first conductor is insulated and provides a signal at the distal end of the shaft, wherein the signal is configured to provide nerve stimulation at the distal end of the shaft of the nerve stimulating bone screw.

2. The nerve stimulating bone screw of claim 2, wherein the dielectric material comprises a polymer material, a ceramic material, or a combination thereof.

3. The nerve stimulating bone screw of claim 3, wherein the polymer material comprises a polyaramide material, a polyimid material, a polyamide material, a polyaryletherketone (PAEK) material, or a combination thereof.

4. The nerve stimulating bone screw of claim 4, wherein the PAEK material comprises polyetherketone (PEK), polyetheretherketone (PEEK), polyetherketoneketone (PEKK), polyetherketoneetherketoneketone (PEKEKK), or a combination thereof.

5. The nerve stimulating bone screw of claim 3, wherein the ceramic material comprises alumina oxide, silica oxide, zirconium oxide, aluminum oxide, or a combination thereof.

6. The nerve stimulating bone screw of claim 1, wherein the shank and the body comprise a metal material.

7. The nerve stimulating bone screw of claim 7, wherein the metal material comprises a pure metal, a metal alloy, or a combination thereof.

8. The nerve stimulating bone screw of claim 8, wherein the pure metal comprises titanium.

9. The nerve stimulating bone screw of claim 8, wherein the metal alloy comprises stainless steel, tungsten carbide, a cobalt-chrome-molybdenum alloy, a titanium alloy, or a combination thereof.

10. The nerve stimulating bone screw of claim 7, further comprising a first insulator around the first conductor.

11. The nerve stimulating bone screw of claim 11, further comprising a second conductor around the first insulator, wherein the nerve stimulating bone screw is configured to transmit a signal via the first conductor and receive at least a portion of the signal via the second conductor.

12. The nerve stimulating bone screw of claim 12, further comprising a second insulator around the second conductor.

13. The nerve stimulating bone screw of claim 1, further comprising a radio frequency identification device within the shaft, the head, or a combination thereof.

14. A method of treating a patient, comprising:
energizing a nerve stimulating bone screw, wherein the nerve stimulating bone screw provides a nerve stimulation signal at a distal end of the nerve stimulating bone screw; and
advancing the nerve stimulating bone screw into tissue.

15. The method of claim 15, further comprising:
determining whether nerve stimulation is occurring.

16. The method of claim 16, further comprising:
ceasing advancement of the nerve stimulating bone screw when nerve stimulation occurs.

17. The method of claim 16, further comprising:
determining whether a desired installation depth is reached.

18. The method of claim 18, further comprising:
de-energizing the nerve stimulating bone screw.

19. A nerve stimulating bone screw, comprising:
a shaft having a continuous thread formed thereon and a distal end;
a head coupled to the shaft;
a first conductor extending along the shaft; and
a second conductor extending along the shaft, wherein the nerve stimulating bone screw is configured to transmit a signal via the first conductor and to receive at least a portion of the signal via the second conductor.

20. A nerve stimulating screwdriver, comprising:
a shaft having a proximal end and a distal end;
a handle coupled to the proximal end of the shaft;
a screw engagement head coupled to the distal end of the shaft;
a first conductor incorporated into the screw engagement head; and
a power source, wherein the power source is configured to provide an electrical signal to the first conductor.

21. The nerve stimulating screwdriver of claim 21, wherein the screw engagement head is configured to engage a nerve stimulating bone screw.

22. The nerve stimulating screwdriver of claim 22, wherein the conductor is configured to transmit the electrical signal to the nerve stimulating bone screw.

23. The nerve stimulating screwdriver of claim 21, further comprising a second conductor incorporated into the screw engagement head.

24. The nerve stimulating screwdriver of claim 24, wherein the nerve stimulating screwdriver is configured to transmit a signal via the first conductor and to receive at least a portion of the signal via the second conductor.

25. The nerve stimulating screwdriver of claim 24, further comprising a processor coupled to the second conductor.

26. A kit, comprising:
at least one nerve stimulating bone screw; and
a nerve stimulating screwdriver, wherein the nerve stimulating screwdriver is configured to deliver an electrical signal to the at least one nerve stimulating bone screw.

27. The kit of claim 27, wherein the at least one nerve stimulating bone screw is configured to transmit the electric signal to a patient.
